(19) United States
(12) Patent Application Publication
(10) Pub. No.: US 2012/0251601 A1
(43) Pub. Date: Oct. 4, 2012

(54) REFRESH DEODORIZING STRIPS

(76) Inventor: Palavi Malik, Jamaica, NY (US)

(21) Appl. No.: 13/432,747

(22) Filed: Mar. 28, 2012

Related U.S. Application Data
(60) Provisional application No. 61/468,242, filed on Mar. 28, 2011.

(51) Int. Cl. A61K 8/02; A61Q 15/00

(52) U.S. Cl. 424/401; 424/65

(57) ABSTRACT

A device for releasing a chemical agent onto a skin of a user, comprising a strip comprising a material that is dissolvable due to heat or moisture under an arm of the user, wherein the chemical agent is at least one of a deodorant or antiperspirant, and wherein the strip encloses the chemical agent in the strip, the chemical agent not being released until the strip dissolves around the chemical agent.

### CROSS-REFERENCE TO RELATED APPLICATION

This application claims the benefit of U.S. Provisional Patent Application Ser. No. 61/468,242, filed on Mar. 28, 2011, the contents of which are herein incorporated by reference in their entirety.

### TECHNICAL FIELD

The embodiments of the present invention relate to deodorizing strips that can be placed under the arm of a user.

### DISCUSSION OF RELATED ART

Known deodorant and/or antiperspirant sticks, sprays or roll-ons, are not easily portable, and are difficult to apply on one's body without drawing attention to the user.

### SUMMARY

The deodorizing strips, according to the embodiments of the present invention, due to their small packaging, are easily portable. Further, the configuration of the deodorizing strips allows them to be applied to a user anywhere, in various situations, without drawing attention to the user. When held under the arm, the deodorizing strips dissolve to release a deodorizing and/or antiperspirant agent. The deodorizing strips begin to dissolve once placed under the arm and may last anywhere from a couple of hours to all day.

A device for releasing a chemical agent onto a skin of a user, according to an embodiment of the present invention, comprises a strip comprising a material that is dissolvable due to heat or moisture under an arm of the user, wherein the chemical agent is at least one of a deodorant or antiperspirant, and wherein the strip encloses the chemical agent in the strip, the chemical agent not being released until the strip dissolves around the chemical agent.

The chemical agent may be in the form of a gel.

A method for applying a chemical agent onto a skin of a user, according to an embodiment of the present invention, comprises placing and holding a strip enclosing the chemical agent in the strip under an arm of the user, wherein the chemical agent is at least one of a deodorant or antiperspirant, dissolving the strip due to heat or moisture under the arm of the user, and releasing the chemical agent onto the skin of the user after dissolving the strip.

### BRIEF DESCRIPTION OF THE DRAWINGS

Exemplary embodiments of the present invention will be described below in more detail, with reference to the accompanying drawings, of which:

FIG. 1 is a front view of a deodorizing strip, according to an embodiment of the present invention; and

FIG. 2 is a front view of deodorizing strip, according to an embodiment of the present invention.

### DETAILED DESCRIPTION OF EXEMPLARY EMBODIMENTS

Exemplary embodiments of the present invention now will be described more fully hereinafter with reference to the accompanying drawings. This invention may, however, be embodied in many different forms and should not be construed as limited to the embodiments set forth herein.

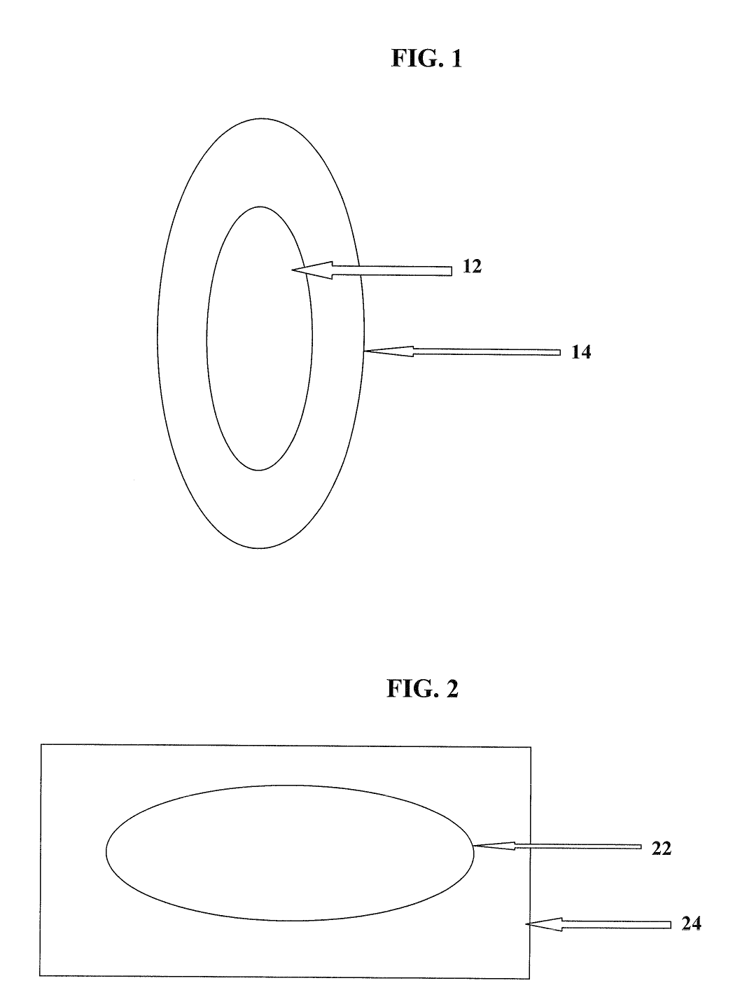

Referring to FIGS. 1 and 2, the deodorizing strips 10, 20 can be sold in a dispenser (not shown), and refills can be sold separately to refill the dispenser when the deodorizing strips run out. After the strips are placed under the arms, a user can feel confident and refreshed. The deodorizing strips give a user the option to repeatedly apply a number of strips at various times.

The deodorant and/or antiperspirant agent 12, 22 is contained within the strip 14, 24, and is not released until the strip 14, 24 dissolves around the deodorant and/or antiperspirant agent 12. 22. The deodorant and/or antiperspirant agent 12, 22 may be, for example, in the form of a gel. The deodorant and/or antiperspirant agent 12, 22 may dissolve once it contacts the skin, or may coat the skin and be absorbed through one's pores. The strip 14, 24 may be a material which can easily dissolve when placed under one's arms, due for example, to heat or moisture under the arms, and which would not irritate the skin of a user.

The deodorizing strips are equally available for men and women.

Although exemplary embodiments of the present invention have been described hereinabove, it should be understood that the present invention is not limited to these embodiments, but may be modified by those skilled in the art without departing from the spirit and scope of the present invention.

What is claimed is:

1. A device for releasing a chemical agent onto a skin of a user, comprising:
a strip comprising a material that is dissolvable due to heat or moisture under an arm of the user, wherein the chemical agent is at least one of a deodorant or antiperspirant, and wherein the strip encloses the chemical agent in the strip, the chemical agent not being released until the strip dissolves around the chemical agent.

2. The device of claim 1, wherein the chemical agent is in the form of a gel.

3. A method for applying a chemical agent onto a skin of a user, comprising:
placing and holding a strip enclosing the chemical agent in the strip under an arm of the user, wherein the chemical agent is at least one of a deodorant or antiperspirant;
dissolving the strip due to heat or moisture under the arm of the user; and
releasing the chemical agent onto the skin of the user after dissolving the strip.

4. The method of claim 3, wherein the chemical agent is in the form of a gel.
